(19) United States
(12) Patent Application Publication
(10) Pub. No.: US 2008/0250511 A1
(43) Pub. Date: Oct. 9, 2008

(54) Preprotachykinin Enhancer Elements

(76) Inventor: Alasdair Mackenzie, Aberdeenshire (GB)

Correspondence Address: MICHAEL BEST & FRIEDRICH LLP, ONE SOUTH PINCKNEY STREET, P O BOX 1806, MADISON, WI, 53701, US

(21) Appl. No.: 12/065,300

(22) PCT Filed: Aug. 22, 2006
(86) PCT No.: PCT/GB2006/003130

§ 371 Date: Feb. 29, 2008

(30) Foreign Application Priority Data
Sep. 1, 2005 (GB) 0517798.5

(51) Int. Cl. C07H 21/00; C12N 5/10; A01K 67/027; C12N 15/89; C12Q 1/68; C12P 19/34

(52) U.S. Cl. 800/8; 536/24.1; 435/325; 800/25; 435/6; 435/91.5; 536/25.3

(57) ABSTRACT

Provided are previously uncharacterised enhancer elements from the preprotachykinin-A (PPTA) which shows transcriptional enhancement activity in Substance P (SP) expressing cells. These are termed ECR1 and ECR2. The invention provides nucleic acids comprising these sequences and variants or fragments thereof, plus methods and materials based thereon, such as transformed host cells, transgenic animals, and screening and expression systems.

### TECHNICAL FIELD

The present invention relates generally to methods and materials relating to tissue specific enhancers of transcription.

### BACKGROUND ART

Substance P (SP) is a neuropeptide (11 amino acids long) widely expressed throughout the nervous system and periphery, including areas such as the sensory neurones of the dorsal root ganglia (DRG)⁶ and the amygdala⁷. It is a member of the tachykinin family, which are a group of neuropeptides that also include NKA, NKB (encoded by the PPTB gene) and Hemokinin (Encoded for by PPTC) (Pennefather et al., 2004). Both SP and NKA are post translationally proteolytically cleaved from a 129 amino acid propeptide encoded by the various mRNA isoforms transcribed from the PPTA (a.k.a. TAC1) gene. SP is encoded by exon 3 of the PPTA gene and NKA is encoded by exon 5.

The physiological effects of the tachykinins are transmitted via a family of three different G-protein-coupled receptors called NK-1, NK-2 and NK-3 that display distinct binding preferences for particular tachykinins (Pennefather et al., 2004). Thus, NK1 displays a distinct preference for the binding of SP (Maggi, 1995).

SP is secreted from sensory neurones which are cells of the peripheral nervous system that are critical to normal inflammatory response and sensations of pain. SP is produced from sensory neurones in response to injury and infection and acts as potent pro-inflammatory cytokine following injury⁸. Levels of sensory neurone-derived SP are increased in affected tissues during the progression of many inflammatory diseases such as asthma, arthritis and inflammatory bowel disease⁸.

SP expressed in the amygdala is involved in modulating mood⁹. Significantly elevated levels of SP are released in the medial amygdaloid nucleus (MeA) in response to stress in rats and it has also been shown that chronic stress increases the transcription of PPTA in the MeA.⁷ Furthermore, it was shown that both the NK1 receptor and its ligand SP are expressed at high levels in the amygdala (Ribeiro-da-Silva and Hokfelt, 2000; Yao et al., 1999). It has been observed that NK1 and SP knockout mice display less fear and anxiety related behaviour and are more aggression than wild type mice (Bilkei-Gorzo et al., 2002; De Felipe et al., 1998). Application of NK1 antagonists has been shown to reduce anxiety both in rodents¹⁰ and in chronically depressed human patients¹¹.

Additionally, the possibility that PPTA may be involved in depressive disorders was suggested by the results of an expanded convergent functional genomic screen that highlighted the possible role of the PPTA gene in contributing to bipolar related disorders (Ogden et al., 2004).

Despite the known importance of the role of the expression of SP in the amygdala in modulating fear and anxiety related behaviour virtually nothing is known about the tissue specific or inducible regulatory systems that control the transcription of the PPTA gene in this critical region of the brain or in other tissues.

Previous studies to define these regulatory systems have used transformed cell lines (Quinn et al., 2000) and organotypic cultures (Hilton et al., 2004; Walker et al., 2000) to describe the presence of regulatory elements within 3.3 kb of the PPTA start site (Quinn et al., 2000). However, transgenic analysis using this 3.3 kb failed to induce any specific marker gene expression in any cells of the amygdala that also expressed PPTA or substances indicating that these putative regulatory elements were not functional in the amygdala in vivo (Harmar et al., 1993; Millward-Sadler et al., 2003).

Thus it can be seen that the provision of materials and methods relating to the regulatory systems that control the transcription of the PPTA gene would provide a contribution to the art.

### DISCLOSURE OF THE INVENTION

The present invention provides previously uncharacterised, clinically relevant, enhancer sequences. These are capable of supporting tissue specific gene expression in the medial amygdaloid nucleus and sensory ganglia.

Briefly, the inventors used comparative genomics to detect the enhancers responsible for driving the complex tissue specific expression of the preprotachykinin-A (PPTA) gene that plays a critical role in modulating nociception, inflammation and mood³⁻⁵. Comparisons restricted to mammalian species were unsuccessful due to high levels of conserved genomic “noise”. However, by comparing the human and chicken genomes it was possible to detect and characterise two long distance enhancers that drive the tissue specific expression of the PPTA in SP expressing cells of sensory neurones and the amygdala. These enhancers are termed “ECR1” and “ECR2” herein. ECR1 was located 180 kb 5′ of the human PPTA gene transcriptional start site. ECR2 was located 216 kb 5′ from the PPTA transcriptional start site.

Further bioinformatics analysis using TRANSFAC databases indicated that the ECR1 element contained multiple and highly conserved consensus binding sequences of transcription factors such as MEIS1 and the glucocorticoid receptor (GR). The results of immunohistochemical analysis of transgenic lines were consistent with the hypothesis that the MEIS1 transcription factor interacts with and maintains ECR1 activity in vivo.

The provision of these enhancers permits inter alia the detailed dissection of the gene regulatory systems that control PPTA expression during anxiogenic stimulation and the progression of inflammatory disease and depression. They also have utility in providing tissue specific expression in a variety of contexts. Further aspects of the invention will now be discussed.

Nucleic Acids

In one aspect the invention provides an isolated DNA molecule comprising an enhancer element which shows transcriptional enhancement activity in SP expressing cells.

Nucleic acids according to the present invention may be provided isolated and/or purified from their natural environment, in substantially pure or homogeneous form, or free or substantially free of other nucleic acids of the species of origin. Where used herein, the term “isolated” encompasses all of these possibilities.

Nucleic acids of the invention may be wholly or partially synthetic. In particular they may be recombinant in that nucleic acid sequences which are not found together in nature (do not run contiguously) have been ligated or otherwise combined artificially. Alternatively they may have been synthesised directly e.g. using an automated synthesiser.

The enhancer elements of the present invention show greater activity in SP expressing cells (e.g. those of the sensory neurones and the amygdala) as compared with non-SP expressing cells (for example NIH 3T3 cells, Hela cells, muscle cells, hepatic liver cells, any one or more of which may be used as a control). This can be assayed using cell lines and reporter genes as described herein.

By “enhancer element” is meant a cis-acting sequence, the activity of which increases the utilisation of a core promoter hence increases the level of transcription of a gene or other ORF operably linked to the promoter, when in the presence of appropriate trans-acting protein factors. Typically such enhancers can function in either orientation and in any location (upstream or downstream) relative to the promoter. The level of transcriptional (i.e. promoter) activity is quantifiable for instance by assessment of the amount of mRNA produced by transcription from the promoter or by assessment of the amount of protein product produced by translation of mRNA produced by transcription from the promoter.

By “promoter” is meant a sequence of nucleotides from which transcription may be initiated of DNA operably linked downstream (i.e. in the 3′ direction on the sense strand of double-stranded DNA). A “core” promoter will generally not show tissue specificity.

By “operably linked” is meant joined as part of the same nucleic acid molecule, suitably positioned and oriented for transcription to be initiated from the promoter.

Preferred SP expressing cells that support the activity of ECR1 in vivo are those of the medial amygdaloid nucleus (MeA), the central amygdaloid (CeM) nucleus and the zona incerta of the thalamus. The MeA plays a critical role in the modulation of emotions such as fear, anxiety and anger (Hasenohrl et al., 2000). The CeM is known as the nociceptive amygdala as it is involved in the processing of noxious sensory information (Neugebauer V, Li W, Bird G C and Han J S (2004) The amygdala and persistent pain. Neuroscientist 10:221-34.). The amygdala is a key part of the limbic system and is comprised of many sub-nuclei which can be separated into four distinct functional groups that are characterised by their efferent and afferent connections and include central, medial, cortical and basomedial, lateral and basolateral nuclei (Misslin, 2003). All of the nuclei forming the amygdala have been shown to be involved in the generation, regulation and expression of anxiety/stress responses (Sajdyk et al., 2004). As demonstrated in the Examples below, below ECR1 drives expression of the LacZ gene into the SP expressing cells of the MeA in several transgenic lines.

Other preferred SP expressing cells are sensory neurones of the dorsal root ganglia (DRG). As demonstrated in the Examples below, below ECR2 drives expression of the LacZ gene in such neurones in transgenic lines.

In one embodiment the enhancer element comprises:

(i) any ECR nucleotide sequence shown in FIG. 1 (ECR1) or FIG. 2 (ECR2),

(ii) a variant nucleotide sequence of any ECR nucleotide sequence shown in FIG. 1 (ECR1) or FIG. 2 (ECR2) sharing at least 65, 70, 80, 90, 95, or 99% identity with a region equal to or at least 100, 120, 140, 160, 180, 200, or 220 contiguous nucleotides shown therein, or

(iii) a fragment of any ECR nucleotide sequence shown in FIG. 1 (ECR1) or FIG. 2 (ECR2) at least 100, 120, 140, 160, 180, 200, or 220 contiguous nucleotides therein, in each case showing transcriptional enhancement activity in SP expressing cells.

As can be seen in FIG. 1, ECR1 exhibits 74% sequence conservation over 217 base pairs between chicken and human and is highly conserved amongst several species.

As can be seen in FIG. 2, ECR2 exhibits 68% sequence conservation over 184 base pairs between chicken and human and is highly conserved amongst several species.

The enhancer element may comprise, consist or consist essentially of any of the sequences, variants or fragments described above. Preferably the enhancer element is less than 300 nucleotides long.

Embodiments of the invention relating to variants and fragments will now be discussed in more detail.

Variants and Fragments

Generally speaking variants may be:

(i) naturally occurring nucleic acids having sequences which are set out in, or may be isolated in the light of, the disclosure herein. The may include homologues or allelic variants of the sequences set out in FIG. 1 or 2. Common mutational changes which give rise to allelic variants are generally ascribed to natural deletions, additions, or substitutions of nucleotides. Each of these types of changes may occur alone, or in combination with the others, one or more times in a given sequence.

(ii) artificial derivatives, which can be prepared by the skilled person in the light of the present disclosure. Such derivatives may be prepared, for instance, by site directed or random mutagenesis, or by direct synthesis. Preferably the variant nucleic acid is generated either directly or indirectly (e.g. via one or more amplification or replication steps) from an original nucleic acid having all or part of the sequence shown in FIG. 1 or FIG. 2.

The percent identity of two nucleotide sequences can be determined by visual inspection and mathematical calculation, or more preferably, the comparison is done by comparing sequence information using a computer program.

An exemplary, preferred computer program is the Genetics Computer Group (GCG; Madison, Wis.) Wisconsin package version 10.0 program, ‘GAP’ (Devereux et al., 1984, Nucl. Acids Res. 12: 387). The preferred default parameters for the ‘GAP’ program includes: (1) The GCG implementation of a unary comparison matrix (containing a value of 1 for identities and 0 for non-identities) for nucleotides (2) a penalty of 50 for each gap and an additional penalty of 3 for each symbol in each gap for nucleotide sequences; (3) no penalty for end gaps; and (4) no maximum penalty for long gaps.

Variants of the invention may be substantially homologous to the nucleotide sequences of FIG. 1 or FIG. 2, and hybridise to the complements thereof. One common formula for calculating the stringency conditions required to achieve hybridization between nucleic acid molecules of a specified sequence homology is (Sambrook et al., 1989): Tₘ=81.5° C.+16.6 Log [Na+]+0.41(% G+C)−0.63(% formamide)−600/#bp in duplex. As an illustration of the above formula, using [Na+]=[0.368] and 50-% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such a sequence would be considered substantially homologous to the nucleic acid sequence of the present invention.

Preferred variants and fragments include one or more of the shaded conserved subsequences shown in FIG. 1 or FIG. 2 i.e. preferably the nucleotides showing sequence identity between the variant and a reference sequence in FIG. 1 or FIG. 2 are those identified as conserved in those Figures.

Preferably variants and fragments include at least 1,2,3,4, or 5 of the ‘TRANSFAC’ sites identified herein (see FIG. 1 and FIG. 3).

In the case of ECR1 variants, there is preferably at least 1 MEIS1 binding site (TGACAG), more preferably 1, 2, 3 or 4 MEIS1 binding sites. MEIS1 is transcription factor of the TALE class of homeodomain proteins that was first discovered as a result of its connection with incidences of myeloid leukaemia. MEIS1 was subsequently found to be expressed in the fore brain and eyes of the developing embryo. The MEIS1 gene has also been shown to be expressed in the post natal brain in areas that include the amygdala.

In the case of ECR1 variants, there is preferably at least 1 GR binding site.

The invention further provides a method of producing a derivative enhancer element, the method comprising the step of modifying any of the sequences disclosed in FIG. 1 or FIG. 2 or substantially homologous variants thereof from other species.

Where any nucleic acid (or nucleotide sequence) of the invention is referred to herein, the complement of that nucleic acid (or nucleotide sequence) will also be embraced by the invention. The ‘complement’ in each case is the same length as the reference, but is 100% complementary thereto whereby each nucleotide is base paired to its counterpart i.e. G to C, and A to T or U. Likewise the reverse complement is also embraced.

In the following, unless context demands otherwise, the term “enhancer element” should be understood to mean any enhancer element of the invention discussed above, including (without limitation) any of the sequences, variants, fragments and complements showing transcriptional enhancement activity in SP expressing cells.

Nucleic Acid Constructs and Vectors

In one aspect there is provided an isolated DNA molecule comprising an enhancer element and a core promoter. The transcriptional activity of the core promoter is increased in SP expressing cells by the enhancer element.

Possible promoters will be well known to those skilled in the art, and include promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40); from heterologous or homologous mammalian promoters, e.g. TK and Pgk-1 core promoters, the actin promoter or an immunoglobulin promoter, and from heat-shock promoters, provided such promoters are compatible with the host cell systems.

In one aspect there is provided an isolated DNA molecule comprising an enhancer element, a core promoter, and operably linked to the core promoter, a target sequence which it is desired to transcribe. The core promoter and target sequence may be heterologous. The level of transcription of the target sequence is increased in PPTA expressing cells by the enhancer element.

A “heterologous” region or domain of a DNA construct is an identifiable segment of DNA within a larger DNA molecule and\or (where context demands it) in a host cell with which it is not found in nature. For example the enhancer may be flanked or followed by DNA that does not flank or follow the enhancer in the genome of the source organism.

Possible target sequences are discussed in more detail below, but include non-coding sequences (e.g. for purposes of down-regulation of target genes) or coding sequences (e.g. comprising an open reading frame having at least one exon of a protein coding sequence). Coding sequences may be, for example, marker genes, therapeutic genes, or any other gene it is desired to express in a tissue specific manner.

The invention also provides a method of transcribing a target sequence by operably linking it to a DNA molecule comprising a core promoter and an enhancer element and introducing it into a cell or in vitro transcription system having appropriate trans acting factors e.g. to achieve transcription in a tissue specific manner in SP producing cells. Preferably the target sequence is expressed as an encoded protein in an appropriate system.

In one aspect of the present invention, the nucleic acid including the enhancer element described above is in the form of a recombinant and preferably replicable vector.

Thus this aspect of the invention provides a gene construct, preferably a replicable vector, comprising an enhancer element, a core promoter, and operably linked to the core promoter, a target sequence which it is desired to transcribe.

“Vector” is defined to include, inter alia, any plasmid, cosmid, yeast artificial chromosome, P1 artificial chromosome, bacterial artificial chromosome, mammalian artificial chromosome or phage, in double- or single-stranded, linear or circular, form which may or may not be self transmissible or mobilizable, and which can transform a prokaryotic or eukaryotic host either by integration into the cellular genome or exist extrachromosomally (e.g. autonomous replicating plasmid with an origin of replication).

Generally speaking, those skilled in the art are well able to construct vectors and design protocols for recombinant gene expression. Suitable vectors can be chosen or constructed, containing, in addition to the elements of the invention described above, appropriate regulatory sequences, including promoter sequences, terminator fragments, polyadenylation sequences, marker genes and other sequences as appropriate. For further details see, for example, Molecular Cloning: a Laboratory Manual. 2nd edition, Sambrook et al, 1989, Cold Spring Harbor Laboratory Press or Current Protocols in Molecular Biology, Second Edition, Ausubel et al. eds., John Wiley & Sons, (1995, and periodic supplements).

In mammalian cells, a number of viral-based expression systems may be utilized e.g. adenovirus, SV40 or EBV-based vectors are all well known to those skilled in the art. Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes.

Cells and Cell Lines

For long term study of transcriptional enhancement in mammalian systems, stable expression of a target protein in a cell line is preferred. For example, sequences encoding a target protein can be transformed into PPTA expressing cell lines using expression vectors containing an enhancer element of the invention plus viral origins of replication and/or endogenous expression elements and a selectable marker gene on the same or on a separate vector.

Thus in a further aspect, the present invention provides a host cell, or cell line, containing heterologous nucleic acid of the invention as described above e.g. an enhancer element, a core promoter, and operably linked to the core promoter, a target sequence which it is desired to transcribe. The nucleic acid of the invention may be integrated into the genome (e.g. chromosome) of the cell. Integration may be promoted by inclusion of sequences which promote recombination with the genome, in accordance with standard techniques.

The methods of the invention may thus include introducing a heterologous nucleic acid of the invention as described above (e.g. an enhancer element, a core promoter, and operably linked to the core promoter, a target sequence which it is desired to transcribe) into a host cell and optionally observing transcription or expression of the target sequence.

The introduction, which may (particularly for in vitro introduction) be generally referred to without limitation as “transformation” or “transfection”, may employ any available technique. For mammalian cells without such cell walls, the calcium phosphate precipitation method of Graham and van der Eb, Virology 52:456-457 (1978) can be employed. General aspects of mammalian cell host system transformations have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g, polybrene, polyornithine, may also be used. For various techniques for transforming mammalian cells, see Keown et al., Methods in Enzymology, 185:527 537 (1990) and Mansour et al., Nature 336:348-352 (1988).

Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk- and apr-cells, respectively. (See, e.g., Wigler, M. et al. (1977) Cell 11:223-232; and Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively. (See, e.g., Wigler, M. et al. (1980) Proc. Natl. Acad. Sci. 77:3567-3570; Colbere-Garapin, F. et a (1981) J. Mol. Biol. 150:1-14; and Murry, supra.) Additional selectable genes have been described, e.g., trpB and hisD, which alter cellular requirements for metabolites. (See, e.g., Hartman, S. C. and R. C. Mulligan (1988) Proc. Natl. Acad. Sci. 85:8047-8051.)

Visible markers, e.g., anthocyanins, green fluorescent proteins (GFP) (Clontech, Palo Alto, Calif.), 6 glucuronidase and its substrate β-D-glucuronoside, or luciferase and its substrate luciferin may be used.

These selection genes and markers can be used not only to identify transformants, but also to quantify the amount of transient or stable protein expression attributable to the enhancer element and promoter system (see, e.g., Rhodes, C. A. et al. (1995) Methods Mol. Biol. 55:121-131.)

Therefore in aspects of the invention relating to an enhancer sequence comprised within a nucleic acid further comprising a core promoter, and (operably linked to the core promoter) a target sequence which it is desired to transcribe, the target sequence may be a detectable marker gene as described above or otherwise known in the art. Thus, for example, the enhancers of the invention may be used in the study of biology of inflammatory disease and chronic depression, for example by expression of marker genes or genes designed to manipulate the characteristics of test cells to determine their roles in disease. Marker genes may be used, for example, in vivo, in cell lines, or in the generation of transgenic animals.

Tissue-Specific Expression of Foreign Genes

Thus in one aspect the invention provides a method of labeling or visualising populations or subpopulations of PPTA/SP expressing cells in vivo, which method comprises introducing into the cell (or an ancestor thereof) a nucleic acid comprising an enhancer sequence, a core promoter, and operably linked to the core promoter, a detectable marker gene as described above or otherwise known in the art.

In one embodiment of the method, the SP expressing cell is preferentially labeled or visualised compared to other non-SP expressing cells into which the DNA molecule is also introduced.

Animals

Although much of the forgoing discussion has been concerned with cell-line or in vitro-based assays, the invention disclosed herein also has utility in animal-models.

Thus host cells according to the present invention may be comprised in a transgenic animal, and the present invention further provides a transgenic animal, comprising cells which include a heterologous nucleic acid of the invention e.g. an enhancer element, a core promoter, and operably linked to the core promoter, a target sequence which it is desired to transcribe.

The transgenic organisms of the invention all include within a plurality of their cells the enhancer element of the invention. Since it is possible to produce transgenic organisms of the invention utilizing one or more of the above-described sequences, a general description will be given of the production of transgenic organisms by referring generally to exogenous genetic material. This general description can be adapted by those skilled in the art in order to incorporate the above-described specific DNA sequences into organisms and observe transcriptional enhancement activity by those sequences utilizing the methods and materials described below. For more details regarding the production of transgenic organisms, and specifically transgenic mice, refer to U.S. Pat. No. 4,873,191, issued Oct. 10, 1989 (incorporated herein by reference to disclose methods producing transgenic mice), and to the numerous scientific publications referred to and cited therein.

The exogenous genetic material may be placed in either the male or female pronucleus of the zygote. More preferably, it is placed in the male pronucleus as soon as possible after the sperm enters the egg. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material could then be added to the ovum or the decondensed sperm could be added to the ovum with the exogenous genetic material being added as soon as possible thereafter.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the DNA sequences which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of a gene, in order to insure that one copy is functional. As regards the present invention, there is generally an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art (see above).

Thus the present invention provides methods in which cloned recombinant DNA sequences encoding appropriate membrane targeting sequences may be injected into fertilized mammalian eggs (preferably mouse eggs). The injected eggs are implanted in pseudo pregnant females and are grown to term to provide transgenic mice whose cells include heterologous enhancer sequences of the invention.

Non-human animals of the invention may be homozygous or heterozygous for the fusion polypeptide. Mammalian animals include non-human primates, rodents, rabbits, sheep, cattle, goats, pigs. Rodents include mice, rats, and guinea pigs. Specifically provided are:

(i) Methods of preparing a transgenic animal model incorporating a nucleic acids as described above such as to express a target sequence in SP producing cells of the model, and the transgenic animal made by such methods.

(ii) Methods of producing an F₁ generation by crossing a founder animal of either sex (F₀ generation) with an animal which is non-transgenic in respect of the proteins discussed herein, and is preferably wild-type). The offspring (F₁ generation) may then be screened and those which carry the nucleic acid of the invention are selected.

(iii) Methods of producing an F₂ generation by crossing 2 F₁ animals of appropriate sex. The offspring (F₂ generation) may then be screened and those which carry a nucleic acid of the invention in the appropriate dosage (i.e. hetero or homozygous), are selected.

As described in the Examples below, the present inventors provide transgenic lines that express marker genes under the influence of the ECR1 and ECR2, which have utility, inter alia, in characterising mechanisms linking the dopaminergic and serotoninergic pathways, known to modulate SP expression in the amygdala, to the regulation of SP expression within cells of the amygdaloid nucleus.

Screening

In yet another embodiment, this invention provides a method of screening a compound for pharmacological activity comprising culturing a SP expressing cell transfected with a heterologous DNA molecule as described above (having an enhancer element, promoter, and protein coding sequence) and determining expression of the protein coding sequence in the presence and absence of the compound.

Analogous screens can also be employed in animal models which are transgenic for the enhancer element, promoter, and protein coding sequence, whereby the compound is administered to the transgenic animal.

Trans-Acting Factors and Binding Partners

The invention further provides a method of binding or identifying a trans-acting factor in a sample, which method comprises contacting the sample with an enhancer element of the invention. The trans-acting factor will generally be one which occurs in PPTA/SP expressing cells. This can be done, for example, by “one-hybrid screening”, which is a powerful method to rapidly identify heterologous transcription factors that can interact with a specific regulatory DNA sequence of interest (the bait sequence). In the one-hybrid system, detection is based on the interaction of a transcription factor (prey) with a bait DNA sequence upstream of a reporter gene. cDNA expression libraries are used to produce hybrids between the prey and a strong trans-activating domain see e.g. Sieweke, M. 2000. “Detection of transcription factor partners with a yeast one hybrid screen”. Methods Mol. Biol. 130:59-77.

Gene Therapy

One of the main obstacles preventing the development of cell and tissue specific gene therapies against inflammatory and depressive diseases has been the unavailability of the required cell and tissue specific enhancers that can direct the expression of therapeutic proteins into specific cell types (Chernajovsky et al., 2004).

The use of tissue specific enhancers such as those described herein can permit the design of vectors in which, following application, the products of therapeutic gene are only produced primarily within specific cell types, for examples those expressing SP. This may be used, for example, for the targeted expression and delivery to SP expressing sensory neurones and the medial amygdaloid nucleus. This may have utility in the production of gene therapies designed to reduce the painful inflammatory symptoms associated with inflammatory diseases such as asthma, arthritis and inflammatory bowel disease and depressive anxiety disorders.

Thus in aspects of the invention discussed above concerning DNA molecules comprising an enhancer element, a core promoter, and operably linked to the core promoter, a target sequence which it is desired to transcribe, the target sequence is preferably a therapeutic protein—for example an anti-inflammatory gene product.

Alternatively the target sequence may be one which targets and down-regulates an endogenous gene implicated in a pathological condition when expressed, or over-expressed, in SP expressing cells.

Double stranded RNA (dsRNA) has been found to be even more effective in gene silencing than both sense or antisense strands alone (Fire A. et al Nature, Vol 391, (1998)). dsRNA mediated silencing is gene specific and is often termed RNA interference (RNAi) (See also Fire (1999) Trends Genet. 15: 358-363, Sharp (2001) Genes Dev. 15: 485-490, Hammond et al. (2001) Nature Rev. Genes 2: 1110-1119 and Tuschl (2001) Chem. Biochem. 2: 239-245). The use of RNA interference as an approach to gene therapy is becoming increasingly widespread see e.g. Zang et al. (Genetic Vaccines and Therapy 2005, 3:5).

Accordingly, this aspect of the invention may employ a vector as described above having an enhancer sequence of the invention, and further including a sequence suitable for introducing an siRNA into the cell. In one embodiment, the vector may comprise a nucleic acid sequence in both the sense and antisense orientation, such that when expressed as RNA the sense and antisense sections will associate to form a double stranded RNA. This may for example be a long double stranded RNA (e.g., more than 23nts) which may be processed in the cell to produce siRNAs (see for example Myers (2003) Nature Biotechnology 21:324-328). Alternatively, the double stranded RNA may directly encode the sequences which form the siRNA duplex. These vectors and RNA products may be useful for example to inhibit de novo production of pathological polypeptides in SP producing cells.

The invention further provides such vectors and DNA molecules for use in a method of treatment (e.g. of a human or animal in need of the same), for example of an inflammatory disease or a depressive anxiety disorder.

The invention further provides use of such DNA molecules in the preparation of a medicament (e.g. a therapeutically effective dose) for example for the treatment of an inflammatory disease or a depressive anxiety disorder.

Expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors to express the DNA molecules of the invention (See, e.g., Sambrook, supra; and Ausubel, supra.)

Example gene therapy vectors for use in the method of this invention include retroviral or episomal vectors expressing particular desired genes under the control of the promoter and/or the supplemental control sequences disclosed herein (see, e.g., Axel, et al., U.S. Pat. No. 4,399,216, and Pastan, et al., U.S. Pat. No. 5,166,059, both incorporated herein by reference). Delivery systems as contemplated herein include both viral and liposomal delivery systems (see, e.g., Davis, et al., U.S. Pat. No. 4,920,209, incorporated herein by reference).

Detection of Disease Susceptibility

Individual susceptibility to chronic depression and anxiety related disorders has been shown to have strong, although complex, genetic components (Blumenthal, 2005; Brant and Shugart, 2004; Malhi et al., 2000; Spector and MacGregor, 2004). Because of their relative ease of identification the genetics of susceptibility to these diseases is most often examined from the perspective of exonic sequence and polymorphisms that alter or curtail the biochemical activity of particular proteins. For example, a recent study has demonstrated a link between the PPTA gene and bipolar depression (Ogden et al., 2004).

In comparison, little has been done to determining the role that gene mis-expression may play in the development and exacerbation of psychiatric disease.

However, recent microarray based expression profiling has demonstrated that gene expression levels between individuals of the same species can vary enormously (Morley et al., 2004). In addition, this variance in expression has been shown to be governed by a strong heritable component that, in many cases, is cis-acting (Pastinen and Hudson, 2004; Wittkopp et al., 2004). It is therefore highly likely that the incidence of many diseases is related to differences within the regulatory systems that control the expression of key genes as opposed to mutations within the coding sequences of these genes, and this includes polymorphism/mutation induced changes in the expression of neuropeptides in the amygdala which may lead to to susceptibility to anxiety and depressive disorders (MacKenzie and Quinn, 2004).

Owing to the clinical relevance of the genes controlled by ECR1 and ECR2, these sequences may be used as screening targets for polymorphisms or other markers associated with disease susceptibility.

Therefore in one embodiment the invention provides a method of screening for, or identifying, a marker (e.g. polymorphism or mutation) associated with anxiety or a depressive disorder, which method comprises:

(i) amplifying all or part of an enhancer sequence of the invention from a genomic nucleic acid sample,

(ii) determining the presence or absence of the marker in the enhancer sequence.

Preferably amplification will employ a PCR primer pair which comprises first and second primers which hybridise to DNA in regions within or flanking an ECR1 or ECR2 genomic sequence described herein such as to amplify all or part of the sequence. Preferably the primers amplify less than 300, more preferably less than 250, 200, 150, 100, 50, 25, or 14 nucleotides.

“Amplification,” as used herein, relates to the production of additional copies of a nucleic acid sequence. Amplification is generally carried out using polymerase chain reaction (PCR) technologies well known in the art. (See, e.g., Dieffenbach, C. W. and G. S. Dveksler (1995) PCR Primer, a Laboratory Manual, Cold Spring Harbor Press, Plainview, N.Y., pp. 1-5.)

In another aspect, polymorphisms may be identified using a microarrays. Microarrays may be prepared, used, and analyzed using methods known in the art (see, e.g., Brennan, T. M. et al. (1995) U.S. Pat. No. 5,474,796; Schena, M. et al. (1996) Proc. Natl. Acad. Sci. 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. 94:2150-2155; and Heller, M. J. et al. (1997) U.S. Pat. No. 5,605,662.) Exemplary methods of detecting or screening polymorphisms or mutations are described for example in WO2004/046381 (University of Aberdeen).

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1 shows a 241 nucleotide human ECR1 sequence, as compared with homologues from other species. Likely transcription factor binding sites (according to TRANSFAC) are noted.

FIG. 2 shows a 225 nucleotide human ECR2 sequence, as compared with homologues from other species.

FIG. 3 shows the ECR2 sequence, as compared with homologues from other species, with areas of conservation with the chicken sequence highlighted. Likely transcription factor binding sites (according to TRANSFAC) are noted.

### EXAMPLES

Materials and Methods.

Bioinformatic analysis. Chicken sequence for analysis was recovered from the Ensembl web site (http://www.ensembl.org/). Genome comparisons (phylogenetic footprinting) using these recovered sequences were carried out using VISTA (http://pipeline.lbl.gov/cgi-bin/gateway2) and the ECR browser (http://ecrbrowser.dcode.org/) (Ovcharenko et al., 2004). Detection of transcription factor (TF) binding consensus sequences was carried out using the web based MATCH (http://www.gene-regulation.com/cgi-bin/pub/programs/match/bin/match.cgi) programme that use the publicly available TRANSFAC 6.0 database (http://www.gene-regulation.com/cgi-bin/pub/databases/transfac/search.cgi.)

Cloning of sequences; The MHR1, MHR2, ECR1 and ECR2 sequence used in this study were isolated from human placental DNA using high fidelity PCR (Expand HiFi system, Roche) using the following oligos; MHR1_FOR; CAGAAAACTAGTCAGGTGTG, MHR1_REV; CAGAACCGGTTAAAGTTATTAGT, MHR2_FOR; GGTTGGGCCCTTTAAAGAACATTCTAA, MHR2_REV; AAGACTAAGAATTCAAACTCATGTTAA, ECR1_FOR; TCAAGATAATTTTGCCATGCTG, ECR1_REV; GATCGGTATAGCAAACCACCA, ECR2_FOR TTTTGGGAGAATGGAAGTGG, ECR2_REV TGGCTTGGGGTAATCTTTTT (MWG). All products were blunt end cloned into pGEM3z (Promega) and sequenced (ABI 377) to determine amplification fidelity. PCR products were then ligated into the well characterized reporter construct p1230²⁰ to produce the plasmid constructs MHR1-hβg-LacZ, MHR2-hβg-LacZ, pECR1-hβg-LacZ and pECR2-hβg-LacZ. p1230 has been used successfully many times in the past and the human β-globin promoter has been demonstrated to be incapable of supporting any degree of consistent tissue specific expression of the LacZ gene on its own within transgenic animals (MacKenzie et al., 1997; MacKenzie and Quinn, 1999; Millward-Sadler et al., 2003; Yee and Rigby, 1993). These constructs were then linearized using NotI and ApaI digestion and gel purified for pronuclear microinjection.

Transgenic analysis; Construct DNA was microinjected into 1-cell C57/BL6xCBA F1 mouse embryos at a concentration of 2-4 ng/μl⁻¹ as described (Hogan, 1995). Surviving embryos were oviduct transferred into pseudopregnant CD1 host mothers (Hogan, 1995). All microinjected embryos were allowed to go to term to generate transgenic lines.

Histological analysis; Transgenic mice from each line were humanely sacrificed by halothane overdose and perfused using 4% paraformaldehyde (PFA) in phosphate buffered saline (PBS). These mice were dissected to recover brain, spinal cord and dorsal root ganglia. Tissues were further fixed in 4% in PFA (PBS) for 2 hours. Whole brains were then sectioned coronally into 4 mm slices. All tissues were then stained using 5-bromo-4-chloro-3-indolyl-beta-D-galactopyranoside (X-gal) solution for 4-12 hours as described (Hogan, 1995). X-gal stained tissues were then either prepared for vibratome sectioning or for analysis by immunohistochemistry. For vibratome sectioning tissues were impregnated with increasing concentrations of sucrose (to 20%) in PBS at 4° C. Tissues were then impregnated with BSA/Gelatin solution (0.5% gelatin, 20% sucrose, 15.5% bovine serum albumen (BSA)) overnight at 4° C. BSA/Gelatin embedded tissue was then hardened by addition of one tenth of a volume of 25% gluteraldehyde. 50-100 um sections were then cut on a Vibratome series 1000. Sections were mounted on glass slides and photographed under light field or phase contrast illumination.

Immunohistochemical analysis; For immunohistochemistry analysis X-gal stained tissues were fixed overnight in 4% paraformaldehyde and impregnated with ascending concentrations of sucrose (to 20%) in PBS. Sucrose impregnated tissues were snap frozen in liquid nitrogen and mounted for sectioning in OCT compound. 10-micron sections were cut on a cryotome and mounted on polysine slides. Sections were pre-incubated in 5% donkey serum and 10% BSA in PBS for one hour then washed 3 times in PBS. Sections were then incubated for 48 hrs at 4° C. with 1:5000 dilution of Rabbit anti-SP antibody (Chemicon) or 1:5000 rabbit anti MEIS1 (A king gift from Arthur Blumberg). Sections were then washed in PBS×5 and incubated with Alexa fluor 488 donkey anti-rabbit IgG secondary antibody. Following 3 washes in PBS sections were mounted in Vectasheild with DAPI (Vector labs) and either photographed under fluorescent microscopy or using a Ziess confocal microscope set to optically section 1 μm slices.

### Example 1

### Comparative Analysis of Mammalian Genomes

We have previously demonstrated using transgenic analysis of a 380 kb YAC construct containing the PPTA gene fused to a LacZ reporter, that the elements required for expression of the PPTA gene in neuronal tissues, that include the amygdala, lay within 140 kb 3′ or 240 kb 5′ of the PPTA transcriptional start site (MacKenzie et al., 2000; MacKenzie and Quinn, 2002).

In order to determine the locations and identity of the enhancers responsible for driving expression of the PPTA gene in the amygdala within this 380 kb we carried out genome comparisons using the ECR browser (Ovcharenko et al., 2004) set to detect regions of significant conservation between chicken, mouse, rat, dog and human genome sequences surrounding the PPTA gene. Comparing mammalian sequence alone led to the identification of upwards of 40 identifiable peaks of genome homology (greater that 70% over 100 bp) within the 380 kb of the human genome contained within the previously analysed YAC construct (MacKenzie et al., 2000; MacKenzie and Quinn, 2002) suggesting that the expression of the PPTA gene either depended on many different enhancers or that the peaks of conservation observed represented high levels of conserved sequence with little tissue specific influence on transcription.

### Example 2

### Chicken-Human Genome Comparisons

Comparative analysis of 380 kb of the human and chicken genome corresponding to the sequence contained within the previously analysed 380 kb PPTA containing YAC construct previously analysed (MacKenzie et al., 2000; MacKenzie and Quinn, 2002) succeeded in revealing two evolutionary conserved regions (ECR) of significant conservation within 240 kb 5′ of the PPTA gene.

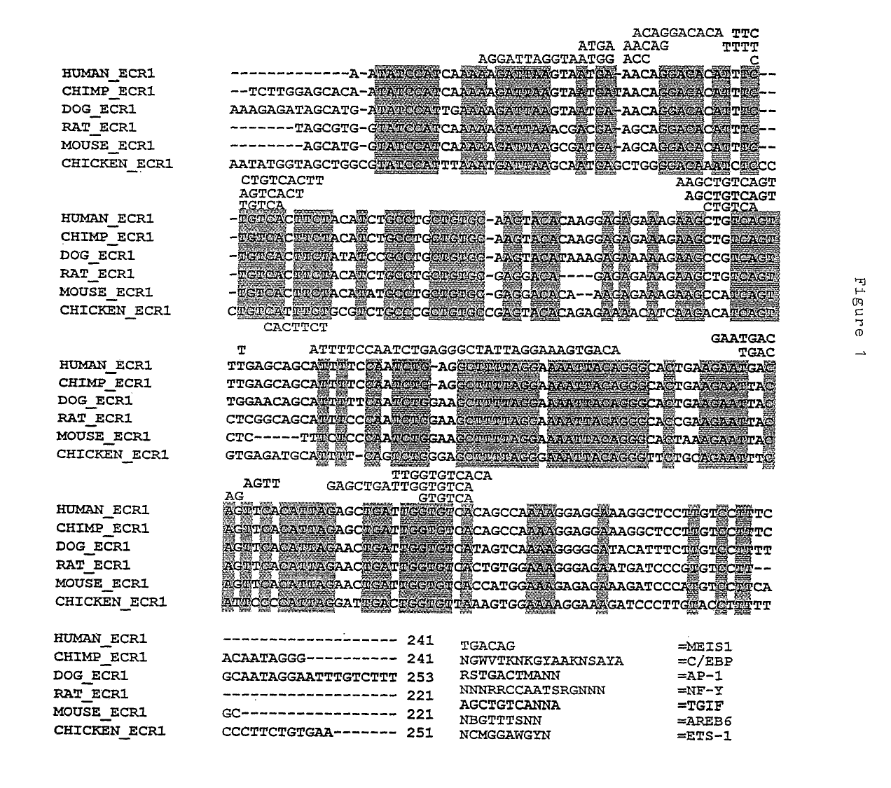

The closest sequence to the PPTA locus, evolutionary conserved region 1 (ECR1) exhibited 74% sequence conservation over 217 base pairs between chicken and human and was located 180 kb 5′ of the human PPTA gene transcriptional start site (FIG. 1).

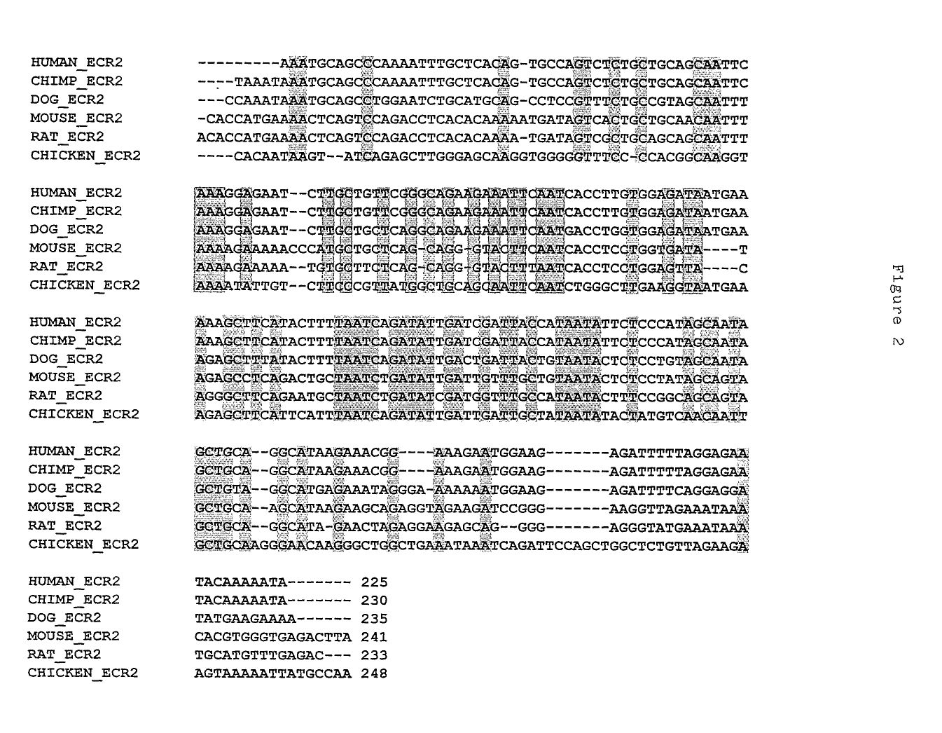

The second sequence, ECR2, exhibited 68% conserved over 184 base pairs and was located 216 kb 5′ from the PPTA transcriptional start site (FIG. 2).

The persistent cis-linkage of these remote and highly conserved sequences with the PPTA gene, despite 310 million years of evolutionary divergence (Wallis et al., 2004) and strong evidence for the occurance of much higher chromosome rearrangement rates between species than was first suspected (Mackenzie et al., 2004) suggested that ECR1 and ECR2 may be responsible for supporting the expression of the PPTA gene.

### Example 3

### Transgenic Analysis of ECR1

We used high fidelity PCR to isolate the human ECR1 sequence and cloned it into a plasmid containing the bacterial LacZ marker gene linked to the human β-globin promoter (p1230) to form pECR1-hβg-LacZ. Linearised pECR1-hβg-LacZ plasmid was micro-injected into 1 cell mouse embryos to generate pECR1-hβg-LacZ transgenic lines.

Three pECR1-hβg-LacZ transgenic lines that we analysed by whole mount LacZ staining and vibratome sectioning demonstrated a strong and consistent LacZ staining pattern within cells of an area of the brain that co-responded to the medial amygdaloid nucleus. LacZ staining was also detected within the central amygdaloid nucleus and could also be detected within parts of the thalamus corresponding to the zona incerta and a restricted population of cells within the caudate putamen. These areas of the amygdala are entirely consistent with the previously described locations of PPTA expression (Sergeyev et al., 2005), localisation of the SP peptide (Ribeiro-da-Silva and Hokfelt, 2000) and PPTA YAC transgenic lines (MacKenzie et al., 2000) in the amygdala, thalamus and the caudate putamen.

In order to determine whether expression of LacZ from the transgene corresponded to the expression of the SP peptide at at the cellular level we carried out fluorescent immunohistochemistry using antibodies against SP on transgenic tissue sections previously stained with X-Gal. We were able to observe the co-localisation of β-galactosidase activity and the expression of SP within populations of cells of the medial and central amygdaloid nucleus.

We also examined the expression of the ECR2-hβg-LacZ transgene in 3 expressing transgenic lines. All of the lines expressed LacZ in cells of the olfactory bulbs, the olfactory tubercle, populations of cells within the thalamus and hypothalamus, the inferior and superior colliculli and in layer IV-VI of the cerebral cortex which is again entirely consistent with those expression patterns previously published^{7,18,22}.

Significantly, LacZ expression was also detected in specific populations of cells within the dorsal root ganglia. Immunohistochemistry was again used to confirm that the expression patterns of LacZ expressed from the transgene co-localised in cells also to expressing SP indicating that ECR2 drives gene expression in SP expressing sensory neurones.

### Example 4

### TRANSFAC Analysis of ECR1

In order to predict the identity of the TFs that interact with ECR1 we subjected ECR1 sequence to analysis using MATCH; a publicly available web tool that allows detection of known transcription factor consensus binding sequences (matrices) based on TRANSFAC data sets. We examined the presence of transcription factor binding sites using stringent conditions where core binding site similarity equaled 100% (core sim.=1) and where matrix similarity was greater that 90% (mat.sim.>0.9). The core similarity denotes the quality of a match between the most conserved 5 nucleotides of a TF binding matrix and ECR1. The matrix similarity is a score that describes the quality of a match between the entire TF binding matrix and ECR1. The matrices of all the transcription factors in the TRANSFAC database are empirically deduced by electrophoretic shift assays and site selection enrichment assays using random oligos (Fogel et al., 2005).

Using these utilities we were able to detect the binding consensus sequences of many different transcription factors that included the Y-box binding factor nuclear factor Y (NF-Y), CCAAT/enhancer binding protein alpha (C/EBP), ETS-1 (Sun and Loh, 2001), AP-1, TGIF, AREB6 and MEIS1. We detected two highly conserved binding sites for NF-Y is a ubiquitously expressed protein (Schmidt and Schibler, 1995) whose binding activity is known to be cAMP dependent (Cote et al., 2002). Furthermore we were able to detect 3 highly conserved binding sites for the widely expressed transcription factors, AP-1 the activity of whose components; fos and jun, are increased in the amygdala as a result of stress (Arnold et al., 1992; Honkaniemi et al., 1992). Interestingly, many of these transcription factor-binding sites in addition to being highly conserved between mammals and even birds, also occurred several times within the most conserved 217 bp of the ECR1 element. For example, the binding site for the MEIS1 transcription factor protein was detected 4 times within the ECR1 element (see FIG. 1). Considering that this 6 base pair element should only occur once in every 16,384 base pairs the detection of 4 binding sites within 217 base pairs is significant. Furthermore, many of these MEIS1 binding sites such as sites 1 and 2 have been highly conserved within mammals with site 1 being perfectly conserved for more that 310 million years. Interestingly, the mRNA and protein distribution of several of these transcription factors closely corresponded to that of the activity of the human PPTA promoter.

We are able to show that embryos from the YAChPPTALacZ transgenic lines demonstrate PPTA promotor activity in areas of the developing embryo brain that overlap that of both the mRNA and protein expression of the MEIS1 homoebox gene in an area of the brain destined to give rise to the amygdala. Therefore, these transcription factors represent good candidates for controlling the expression of the PPTA gene via binding of the ECR1 enhancer.

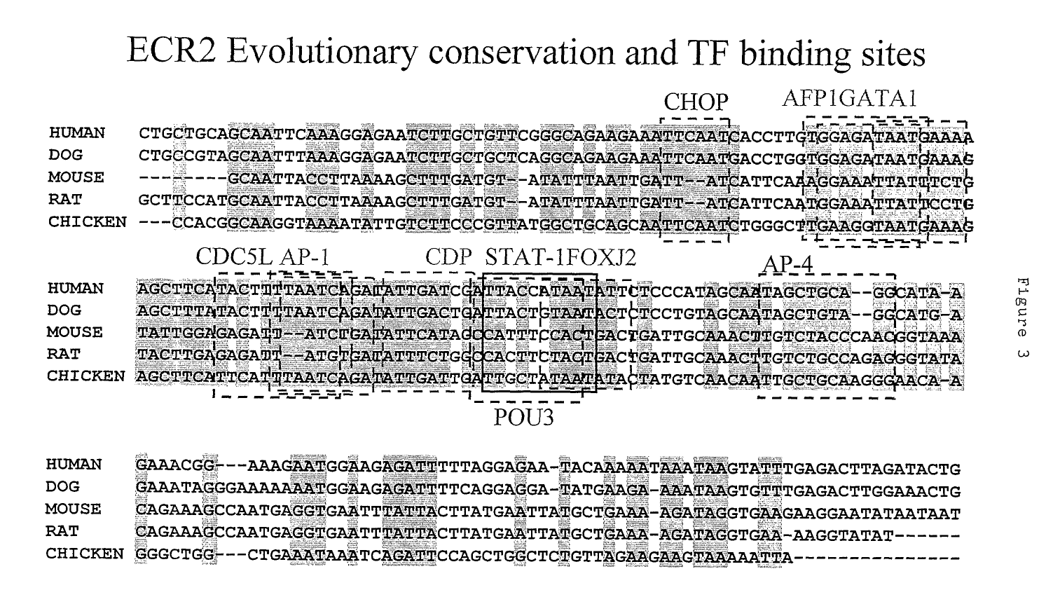

FIG. 3 shows corresponding analysis for ECR2.

### Example 5

### Evidence of the Involvement of MEIS1 in the Regulation of ECR1

In order to test whether any of the transcription factors identified by TRANSFAC analysis were involved in regulating the activity of the ECR1 enhancer we carried out immunohistochemical analysis of brain sections of animals transgenic for the pECR1-hβg-LacZ construct. Using fluorescent confocal microscopy we were able to detect the presence of the MEIS1 transcription factor in a sub population of cells within the amygdala that represented less than 5% of the overall cell population. This population was further divisible into a proportion of cells in which the subcellular localisation of MEIS1 was either cytoplasmic or nuclear. Significantly, we observed that greater than 95% cells of the cells of the central amygdala that contained observable nuclear MEIS1 immunoreactivity also demonstrated β-galactosidase activity. In combination with the presence of 3 highly conserved MEIS1 binding sites within the ECR1 enhancer this observation is entirely consistent with the hypothesis that MEIS1 is involved in modulating aspects of the activity of the ECR1 enhancer in the central amygdala. Similar co-expression was not seen in LacZ staining cells of the thalamus of medial amygdala.

### Example 6

### Evidence of the Involvement of Glucocorticoid Receptor (GR) in the Regulation of ECR1

A number of highly conserved GR binding sites within the ECR1 enhancer have been identified.

In addition, we have used chromatin immunoprecipitation assays on disaggregated mouse amygdala cells using a GR antibody and PCR primers against the most conserved region of the ECR1 to demonstrate that this interaction does indeed occur in the cells.

Thus it appears that the activity of the ECR1 enhancer, an amygdala specific enhancer of the anxiogenic TAC1 gene, is inducable by GR; the receptor of glucocorticoid stress hormones.

The discovery of this relationship between ECR1 and GR points to a molecular mechanisms linking stress with depression and thus may have implications in future anti-depressive drug design.

### REFERENCES:

1. Nobrega, M. A., Ovcharenko, I., Afzal, V. & Rubin, E. M. Scanning human gene deserts for long-range enhancers. Science 302, 413 (2003).

2. Wasserman, W. W. & Krivan, W. In silico identification of metazoan transcriptional regulatory regions. Naturwissenschaften 90, 156-66 (2003).

3. Cao, Y. Q. et al. Primary afferent tachykinins are required to experience moderate to intense pain [see comments]. Nature 392, 390-4 (1998).

4. Zimmer, A. et al. Hypoalgesia in mice with a targeted deletion of the tachykinin 1 gene. Proc Natl Acad Sci USA 95, 2630-5 (1998).

5. Hokfelt, T., Pernow, B. & Wahren, J. SP: a pioneer amongst neuropeptides. J Intern Med 249, 27-40. (2001).

6. Nielsch, U. & Keen, P. Reciprocal regulation of tachykinin- and vasoactive intestinal peptide-gene expression in rat sensory neurones following cut and crush injury. Brain Res 481, 25-30 (1989).

7. Sergeyev, V. et al. Neuropeptide expression in rats exposed to chronic mild stresses. Psychopharmacology (Berl) 178, 115-24 (2005).

8. O'Connor, T. M. et al. The role of SP in inflammatory disease. J Cell Physiol 201, 167-80 (2004).

9. Kramer, M. S. et al. Distinct mechanism for antidepressant activity by blockade of central SP receptors. Science 281, 1640-5. (1998).

10. Ebner, K., Rupniak, N. M., Saria, A. & Singewald, N. SP in the medial amygdala: emotional stress-sensitive release and modulation of anxiety-related behavior in rats. Proc Natl Acad Sci USA 101, 4280-5 (2004).

11. Ranga, K. & Krishnan, R. Clinical experience with SP receptor (NK1) antagonists in depression. J Clin Psychiatry 63 Suppl 11, 25-9 (2002).

12. Leslie, T. A., Emson, P. C., Dowd, P. M. & Woolf, C. J. Nerve growth factor contributes to the up-regulation of growth-associated protein 43 and preprotachykinin A messenger RNAs in primary sensory neurons following peripheral inflammation. Neuroscience 67, 753-61 (1995).

13. Igwe, O. J. c-Src kinase activation regulates preprotachykinin gene expression and SP secretion in rat sensory ganglia. Eur J Neurosci 18, 1719-30 (2003).

14. Adler, J. E. & Walker, P. D. Cyclic AMP regulates SP expression in developing and mature spinal sensory neurons. J Neurosci Res 59, 624-31 (2000).

15. Bae, S. J. et al. SP induced preprotachykinin-a mRNA, neutral endopeptidase mRNA and SP in cultured normal fibroblasts. Int Arch Allergy Immunol 127, 316-21 (2002).

16. Harmar, A. J. et al. 3.3 kb of 5′ flanking DNA from the rat preprotachykinin gene directs high level expression of a reporter gene in microinjected dorsal root ganglion neurons but not in transgenic mice. Regul Pept 46, 67-9 (1993).

17. Millward-Sadler, S. J. et al. Tachykinin expression in cartilage and function in human articular chondrocyte mechanotransduction. Arthritis Rheum 48, 146-56 (2003).

18. MacKenzie, A., Payne, C., Boyle, S., Clarke, A. R. & Quinn, J. P. The human preprotachykinin-A gene promoter has been highly conserved and can drive human-like marker gene expression in the adult mouse CNS. Mol Cell Neurosci 16, 620-30. (2000).

19. Ovcharenko, I., Nobrega, M. A., Loots, G. G. & Stubbs, L. ECR Browser: a tool for visualizing and accessing data from comparisons of multiple vertebrate genomes. Nucleic Acids Res 32, W280-6 (2004).

20. Yee, S. P. & Rigby, P. W. The regulation of myogenin gene expression during the embryonic development of the mouse. Genes Dev 7, 1277-89. (1993).

21. Mackenzie, A., Miller, K. A. & Collinson, J. M. Is there a functional link between gene interdigitation and multi-species conservation of synteny blocks? Bioessays 26, 1217-24 (2004).

22. Ribeiro-da-Silva, A. & Hokfelt, T. Neuroanatomical localisation of SP in the CNS and sensory neurons. Neuropeptides 34, 256-71 (2000).

23. Consortium, I. H. G. S. Finishing the euchromatic sequence of the human genome. Nature 431, 931-45 (2004).

24. Gregory, S. G. et al. A physical map of the mouse genome. Nature 418, 743-50 (2002).

25. Wallis, J. W. et al. A physical map of the chicken genome. Nature 432, 761-4 (2004).

26. Wasserman, W. W., Palumbo, M., Thompson, W., Fickett, J. W. & Lawrence, C. E. Human-mouse genome comparisons to locate regulatory sites. Nat Genet 26, 225-8. (2000).

27. Woolfe, A. et al. Highly Conserved Non-Coding Sequences Are Associated with Vertebrate Development. PLoS Biol 3, e7 (2004).

28. Vandenbroeck, K. et al. Chromosome 7q21-22 and multiple sclerosis: evidence for a genetic susceptibility effect in vicinity to the protachykinin-1 gene. J Neuroimmunol 125, 141-8 (2002).

29. Hogan, B., Beddington, R., Constantini, F. and Lacy, E. Manipulating the mouse embryo (Cold Spring Harbor Laborotory Press, New York, 1995).

Adell A (2004) Antidepressant properties of SP antagonists: relationship to monoaminergic mechanisms? Curr Drug Targets CNS Neurol Disord 3:113-21.

Arnold F J, De Lucas Bueno M, Shiers H, Hancock D C, Evan G I and Herbert J (1992) Expression of c-fos in regions of the basal limbic forebrain following intracerebroventricular corticotropin-releasing factor in unstressed or stressed male rats. Neuroscience 51:377-90.

Bilkei-Gorzo A, Racz I, Michel K and Zimmer A (2002) Diminished anxiety- and depression-related behaviors in mice with selective deletion of the Tac1 gene. J Neurosci 22:10046-52.

Blumenthal M N (2005) The role of genetics in the development of asthma and atopy. Curr Opin Allergy Clin Immunol 5:141-5.

Bosker F J, Westerink B H, Cremers T I, Gerrits M, van der Hart M G, Kuipers S D, van der Pompe G, ter Horst G J, den Boer J A and Korf J (2004) Future antidepressants: what is in the pipeline and what is missing? CNS Drugs 18:705-32.

Brant S R and Shugart Y Y (2004) Inflammatory bowel disease gene hunting by linkage analysis: rationale, methodology, and present status of the field. Inflamm Bowel Dis 10:300-11.

Carrasco G A and Van de Kar L D (2003) Neuroendocrine pharmacology of stress. Eur J Pharmacol 463:235-72.

Chernajovsky Y, Gould D J and Podhajcer O L (2004) Gene therapy for autoimmune diseases: quo vadis? Nat Rev Immunol 4:800-11.

Coplan J D and Lydiard R B (1998) Brain circuits in panic disorder. Biol Psychiatry 44:1264-76.

Cote F, Schussler N, Boularand S, Peirotes A, Thevenot E, Mallet J and Vodjdani G (2002) Involvement of NF-Y and Sp1 in basal and cAMP-stimulated transcriptional activation of the tryptophan hydroxylase (TPH) gene in the pineal gland. J Neurochem 81:673-85.

De Felipe C, Herrero J F, O'Brien J A, Palmer J A, Doyle C A, Smith A J, Laird J M, Belmonte C, Cervero F and Hunt S P (1998) Altered nociception, analgesia and aggression in mice lacking the receptor for SP. Nature 392:394-7.

Ebner K, Rupniak N M, Saria A and Singewald N (2004) SP in the medial amygdala: emotional stress-sensitive release and modulation of anxiety-related behavior in rats. Proc Nat Acad Sci USA 101:4280-5.

Fogel G B, Weekes D G, Varga G, Dow E R, Craven A M, Harlow H B, Su E W, Onyia J E and Su C (2005) A statistical analysis of the TRANSFAC database. Biosystems 81:137-54.

Harmar A J, Mulderry P K, al-Shawi R, Lyons V, Sheward W J, Bishop J O and Chapman K (1993) 3.3 kb of 5′ flanking DNA from the rat preprotachykinin gene directs high level expression of a reporter gene in microinjected dorsal root ganglion neurons but not in transgenic mice. Regul Pept 46:67-9.

Hilton K J, Bateson A N and King A E (2004) A model of organotypic rat spinal slice culture and biolistic transfection to elucidate factors that drive the preprotachykinin-A promoter. Brain Res Brain Res Rev 46:191-203.

Hogan B, Beddington, R., Constantini , F. and Lacy, E. (1995) Manipulating the mouse embryo. Cold Spring Harbor Laborotory Press, New York.

Hokfelt T, Pernow B and Wahren J (2001) SP: a pioneer amongst neuropeptides. J Intern Med 249:27040.

Honkaniemi J, Kainu T, Ceccatelli S, Rechardt L, Hokfelt T and Pelto-Huikko M (1992) Fos and jun in rat central amygdaloid nucleus and paraventricular nucleus after stress. Neuroreport 3:849-52.

Huezo-Diaz P, Tandon K and Aitchison K J (2005) The genetics of depression and related traits. Curr Psychiatry Rep 7:117-24.

Kramer M S, Cutler N, Feighner J, Shrivastava R, Carman J, Sramek J J, Reines S A, Liu G, Snavely D, Wyatt-Knowles E, Hale J J, Mills S G, MacCoss M, Swain C J, Harrison T, Hill R G, Hefti F, Scolnick E M, Cascieri M A, Chicchi G G, Sadowski S, Williams A R, Hewson L, Smith D, Rupniak N M and et al. (1998) Distinct mechanism for antidepressant activity by blockade of central SP receptors. Science 281:1640-5.

Mackenzie A, Miller K A and Collinson J M (2004) Is there a functional link between gene interdigitation and multi-species conservation of synteny blocks? Bioessays 26:1217-24.

MacKenzie A, Payne C, Boyle S, Clarke A R and Quinn J P (2000) The human preprotachykinin-A gene promoter has been highly conserved and can drive human-like marker gene expression in the adult mouse CNS. Mol Cell Neurosci 16:620-30.

MacKenzie A, Purdie L, Davidson D, Collinson M and Hill R E (1997) Two enhancer domains control early aspects of the complex expression pattern of Msx1. Mech Dev 62:290-40.

MacKenzie A and Quinn J (1999) A serotonin transporter gene intron 2 polymorphic region, correlated with affective disorders, has allele-dependent differential enhancer-like properties in the mouse embryo. Proc Natl Acad Sci USA 96:15251-5.

MacKenzie A and Quinn J (2002) A yeast artificial chromosome containing the human preprotachykinin-A gene expresses SP in mice and drives appropriate marker gene expression during early brain embryogenesis. Molecular and Cellular Neuroscience 19:72-87.

MacKenzie A and Quinn J P (2004) Post-genomic approaches to exploring neuropeptide gene mis-expression in disease. Neuropeptides 38:1-15.

Maggi C A (1995) The mammalian tachykinin receptors. Gen Pharmacol 26:911-44.

Malhi G S, Moore J and McGuffin P (2000) The genetics of major depressive disorder. Curr Psychiatry Rep 2:165-9.

Millward-Sadler S J, Mackenzie A, Wright M O, Lee H S, Elliot K, Gerrard L, Fiskerstrand C E, Salter D M and Quinn J P (2003) Tachykinin expression in cartilage and function in human articular chondrocyte mechanotransduction. Arthritis Rheum 48:146-56.

Misslin R (2003) The defense system of fear: behavior and neurocircuitry. Neurophysiol Clin 33:55-66.

Morley M, Molony C M, Weber T M, Devlin J L, Ewens K G, Spielman R S and Cheung V G (2004) Genetic analysis of genome-wide variation in human gene expression. Nature 430:743-7.

Ogden C A, Rich M E, Schork N J, Paulus M P, Geyer M A, Lohr J B, Kuczenski R and Niculescu A B (2004) Candidate genes, pathways and mechanisms for bipolar (manic-depressive) and related disorders: an expanded convergent functional genomics approach. Mol Psychiatry 9:1007-29.

Ovcharenko I, Nobrega M A, Loots G G and Stubbs L (2004) ECR Browser: a tool for visualizing and accessing data from comparisons of multiple vertebrate genomes. Nucleic Acids Res 32:W280-6.

Pastinen T and Hudson T J (2004) Cis-acting regulatory variation in the human genome. Science 306:647-50.

Pennefather J N, Lecci A, Candenas M L, Patak E, Pinto F M and Maggi C A (2004) Tachykinins and tachykinin receptors: a growing family. Life Sci 74:1445-63.

Quinn J P, Fiskerstrand C E, Gerrard L, MacKenzie A and Payne C M (2000) Molecular models to analyse preprotachykinin-A expression and function. Neuropeptides 34:292-302.

Ranga K and Krishnan R (2002) Clinical experience with SP receptor (NK1) antagonists in depression. J Clin Psychiatry 63 Suppl 11:25-9.

Ribeiro-da-Silva A and Hokfelt T (2000) Neuroanatomical localisation of SP in the CNS and sensory neurons. Neuropeptides 34:256-71.

Sajdyk T J, Shekhar A and Gehlert D R (2004) Interactions between NPY and CRF in the amygdala to regulate emotionality. Neuropeptides 38:225-34.

Schmidt E E and Schibler U (1995) Cell size regulation, a mechanism that controls cellular RNA accumulation: consequences on regulation of the ubiquitous transcription factors Oct1 and NF-Y and the liver-enriched transcription factor DBP. J Cell Biol 128:467-83.

Sergeyev V, Fetissov S, Mathe A A, Jimenez P A, Bartfai T, Mortas P, Gaudet L, Moreau J L and Hokfelt T (2005) Neuropeptide expression in rats exposed to chronic mild stresses. Psychopharmacology (Berl) 178:115-24.

Spector T D and MacGregor A J (2004) Risk factors for osteoarthritis: genetics. Osteoarthritis Cartilage 12 Suppl A:S39-44.

Sterneck E and Johnson P F (1998) CCAAT/enhancer binding protein beta is a neuronal transcriptional regulator activated by nerve growth factor receptor signaling. J Neurochem 70:2424-33.

Sun P and Loh H H (2001) Transcriptional regulation of mouse delta-opioid receptor gene: role of Ets-1 in the transcriptional activation of mouse delta-opioid receptor gene. J Biol Chem 276:45462-9.

Walker P D, Andrade R, Quinn J P and Bannon M J (2000) Real-time analysis of preprotachykinin promoter activity in single cortical neurons. J Neurochem 75:882-5.

Wallis J W, Aerts J, Groenen M A, Crooijmans R P, Layman D, Graves T A, Scheer D E, Kremitzki C, Fedele M J, Mudd N K, Cardenas M, Higginbotham J, Carter J, McGrane R, Gaige T, Mead K, Walker J, Albracht D, Davito J, Yang SP, Leong S, Chinwalla A, Sekhon M, Wylie K, Dodgson J, Romanov M N, Cheng H, de Jong P J, Osoegawa K, Nefedov M, Zhang H, McPherson J D, Krzywinski M, Schein J, Hillier L, Mardis E R, Wilson R K and Warren W C (2004) A physical map of the chicken genome. Nature 432:761-4.

Wittkopp P J, Haerum B K and Clark A G (2004) Evolutionary changes in cis and trans gene regulation. Nature 430:85-8.

Yao R, Rameshwar P, Donnelly R J and Siegel A (1999) Neurokinin-1 expression and co-localization with glutamate and GABA in the hypothalamus of the cat. Brain Res Mol Brain Res 71:149-58.

Yee S P and Rigby P W (1993) The regulation of myogenin gene expression during the embryonic development of the mouse. Genes Dev 7:1277-89.

1. An isolated DNA molecule comprising an enhancer element which shows transcriptional enhancement activity in Substance P (SP) expressing cells.

2. A DNA molecule as claimed in claim 1 wherein the SP expressing cells are selected from the list consisting of cells of the: medial amygdaloid nucleus; the central amygdaloid nucleus; zona incerta of the thalamus.

3. A DNA molecule as claimed in claim 1 wherein the enhancer element comprises:
(i) any ECR nucleotide sequence shown in FIG. 1 (ECR1) or FIG. 2 (ECR2);
(ii) a variant nucleotide sequence of any ECR nucleotide sequence shown in FIG. 1 (ECR1) or FIG. 2 (ECR2) sharing at least 65, 70, 80, 90, 95, or 99% identity with a region equal to or at least 100, 120, 140, 160, 180, 200, or 220 contiguous nucleotides shown therein; or
(iii) a fragment of any ECR nucleotide sequence shown in FIG. 1 (ECR1) or FIG. 2 (ECR2) at least 100, 120, 140, 160, 180, 200, or 220 contiguous nucleotides therein,
in each case showing transcriptional enhancement activity in SP expressing cells.

4. A DNA molecule as claimed in claim 1 wherein the enhancer element is less than 300 nucleotides long.

5. A DNA molecule as claimed in claim 1 wherein the enhancer element includes one or more of the conserved subsequences shown within FIG. 1 or FIG. 2.

6. A DNA molecule as claimed in claim 1 wherein the enhancer element includes at least 1,2,3,4, or 5 binding sites for a transcription factor selected from the list consisting of: Y-box binding factor nuclear factor Y (NF-Y); CCAAT/enhancer binding protein alpha (C/EBP); ETS-1; AP-1; TGIF; AREB6; and MEIS1.

7. A DNA molecule as claimed in claim 6 wherein the enhancer element includes at least 1, 2, 3 or 4 MEIS1 binding sites.

8. A DNA molecule as claimed in claim 1 wherein the enhancer element includes at least 1 glucocorticoid receptor (GR) binding site.

9. A DNA molecule as claimed in claim 1 further comprising a heterologous core promoter wherein the transcriptional activity of the core promoter is increased in SP expressing cells by the enhancer element.

10. A DNA molecule comprising as claimed in claim 9 further comprising a heterologous target sequence operably linked to the core promoter.

11. A DNA molecule as claimed in claim 10 wherein the target sequence is selected from:
(i) a non-coding sequence for down-regulation of a target gene or
(ii) a coding sequence selected from: a marker gene, a therapeutic gene.

12. A DNA molecule as claimed in claim 11 wherein the therapeutic gene encodes a an anti-inflammatory gene product.

13. A DNA molecule as claimed in claim 11 wherein the non-coding sequence is capable of down-regulating an endogenous target gene in an organism, which endogenous target gene is implicated in a pathological condition when expressed, or over-expressed, in SP expressing cells in that organism.

14. A DNA molecule as claimed in claim 13 wherein the non-coding sequence is suitable for introducing an siRNA into the cell.

15. A DNA molecule as claimed in claim 1 which is a recombinant and optionally replicable vector.

16. A host cell, or cell line, containing a heterologous DNA molecule as claimed in claim 1.

17. A host cell as claimed in claim 16 which is a gamete or zygote.

18. A transgenic animal comprising a host cell as claimed in claim 17.

19. A method for producing a transgenic animal in which a DNA molecule as claimed in claim 1 is injected into a fertilized mammalian egg cell.

20. A method of binding or identifying a trans-acting factor in a sample, which method comprises contacting the sample with a DNA molecule as claimed in claim 1.

21. A method of screening for, or identifying, a marker associated with anxiety or a depressive disorder, which method comprises:
(i) amplifying all or part of an enhancer element sequence disclosed in FIG. 1 (ECR1) or FIG. 2 (ECR2) or substantially homologous variants thereof from other species from a genomic nucleic acid sample,
(ii) determining the presence or absence of the marker in the enhancer sequence.

22. A method of producing a derivative enhancer element, the method comprising the step of modifying any of the enhancer element sequences disclosed in FIG. 1 (ECR1) or FIG. 2 (ECR2) or substantially homologous variants thereof from other species.

23. A method of transcribing a target sequence by operably linking it to the promoter in a DNA molecule as claimed in claim 10 and introducing it into a cell or in vitro transcription system.

24. A method of labeling or visualising populations or su bpopulations of SP expressing cells in vivo, which method comprises introducing into the cell (or an ancestor thereof) a nucleic acid as claimed in claim 11 wherein the coding sequence is a marker gene and observing the expression product of the marker gene.

25. A method of screening a compound for pharmacological activity comprising culturing a host cell as claimed in claim 11 wherein the coding sequence is a marker gene and determining expression of the product of the marker gene in the presence and absence of the compound.


SEQUENCE LISTING (41 sequences)

SEQ ID NO: 1 - DNA, 220 bp, Homo sapiens
aatatccatcaaaaagattaagtaatgaaacaggacacatttctgtcacttctacatctgcctgctgtgcaagtacacaaggagagaaagaagctgtcagtttgagcagcattttccaatctgaggcttttaggaaaattacagggcactgaagaatgacagttcacattagagctgattggtgtcacagccaaaaggaggaaaggctccttgtcctttc

SEQ ID NO: 2 - DNA, 241 bp, Pan sp.
tcttggagcacaatatccatcaaaaagattaagtaatgataacaggacacatttctgtcacttctacatctgcctgctgtgcaagtacacaaggagagaaagaagctgtcagtttgagcagcattttccaatctgaggcttttaggaaaattacagggcactgaagaattacagttcacattagagctgattggtgtcacagccaaaaggaggaaaggctccttgtcctttcacaataggg

SEQ ID NO: 3 - DNA, 253 bp, Canis familiaris
aaagagatagcatgatatccattgaaaagattaagtaatgaaacaggacacatttctgtcacttctatatccgcctgctgtgcaagtacataaagagaaaaagaagccgtcagttggaacagcatttttcaatctggaagcttttaggaaaattacagggcactgaagaattacagttcacattagaactgattggtgtcatagtcaaaagggggatacatttcttgtccttttgcaataggaatttgtcttt

SEQ ID NO: 4 - DNA, 221 bp, Rattus sp.
tagcgtggtatccatcaaaaagattaaacgacgaagcaggacacatttctgtcacttctacatctgcctgctgtgcgaggacagagagaaagaagctgtcagtctcggcagcatttcccaatctggaagcttttaggaaaattacagggcaccgaagaattacagttcacattagaactgattggtgtcactgtggaaagggagaatgatcccgtgtcctt

SEQ ID NO: 5 - DNA, 221 bp, Mus sp.
agcatggtatccatcaaaaagattaagcgatgaagcaggacacatttctgtcacttctacatatgcctgctgtgcgaggacacaaagagaaagaagccatcagtctctttctcccaatctggaagcttttaggaaaattacagggcactaaagaattacagttcacattagaactgattggtgtcaccatggaaagagagaaagatcccatgtccttcagc

SEQ ID NO: 6 - DNA, 251 bp, Gallus gallus
aatatggtagctggcgtatccatttaaatgattaagcaatgagctggggacaaatctcccctgtcatttctgcgtctgcccgctgtgccgagtacacagagaaaacatcaagacatcagtgtgagatgcattttcagtctgggagcttttagggaaattacagggttctgcagaatttcattccccattaggattgactggtgttaaagtggaaaaggaaagatcccttgtacctttttcccttctgtgaa

SEQ ID NO: 7 - DNA, 18 bp, Artificial sequence
  Likely transcription factor binding site (according to TRANSFAC)
aggattaggtaatggacc

SEQ ID NO: 8 - DNA, 10 bp, Artificial sequence
  Likely transcription factor binding site (according to TRANSFAC)
acaggacaca

SEQ ID NO: 9 - DNA, 11 bp, Artificial sequence
  Likely transcription factor binding site (according to TRANSFAC)
ttttagtcact

SEQ ID NO: 10 - DNA, 12 bp, Artificial sequence
  Likely transcription factor binding site (according to TRANSFAC)
ttcctgtcactt

SEQ ID NO: 11 - DNA, 11 bp, Artificial sequence
  Likely transcription factor binding site (according to TRANSFAC)
aagctgtcagt

SEQ ID NO: 12 - DNA, 11 bp, Artificial sequence
  Likely transcription factor binding site (according to TRANSFAC)
agctgtcagtt

SEQ ID NO: 13 - DNA, 16 bp, Artificial sequence
  Likely transcription factor binding site (according to TRANSFAC)
attttccaatctgagg

SEQ ID NO: 14 - DNA, 18 bp, Artificial sequence
  Likely transcription factor binding site (according to TRANSFAC)
gctattaggaaagtgaca

SEQ ID NO: 15 - DNA, 11 bp, Artificial sequence
  Likely transcription factor binding site (according to TRANSFAC)
gaatgacagtt

SEQ ID NO: 16 - DNA, 16 bp, Artificial sequence
  Likely transcription factor binding site (according to TRANSFAC)
gagctgattggtgtca

SEQ ID NO: 17 - DNA, 11 bp, Artificial sequence
  Likely transcription factor binding site (according to TRANSFAC)
ttggtgtcaca

SEQ ID NO: 18 - DNA, 18 bp, Artificial sequence
  CCAAT/enhancer binding protein alpha (C/EBP) consensus sequence
ngwvtknkgyaaknsaya

SEQ ID NO: 19 - DNA, 11 bp, Artificial sequence
  AP-1 consensus sequence
rstgactmann

SEQ ID NO: 20 - DNA, 16 bp, Artificial sequence
  Nuclear factor Y (NF-Y) consensus sequence
nnnrrccaatsrgnnn

SEQ ID NO: 21 - DNA, 11 bp, Artificial sequence
  TGIF consensus sequence
agctgtcanna

SEQ ID NO: 22 - DNA, 10 bp, Artificial sequence
  ETS-1 consensus sequence
ncmggawgyn

SEQ ID NO: 23 - DNA, 225 bp, Homo sapiens
aaatgcagcccaaaatttgctcacagtgccagtctctgctgcagcaattcaaaggagaatcttgctgttcgggcagaagaaattcaatcaccttgtggagataatgaaaaagcttcatacttttaatcagatattgatcgattaccataatattctcccatagcaatagctgcaggcataagaaacggaaagaatggaagagatttttaggagaatacaaaaata

SEQ ID NO: 24 - DNA, 230 bp, Pan sp.
taaataaatgcagcccaaaatttgctcacagtgccagtctctgctgcagcaattcaaaggagaatcttgctgttcgggcagaagaaattcaatcaccttgtggagataatgaaaaagcttcatacttttaatcagatattgatcgattaccataatattctcccatagcaatagctgcaggcataagaaacggaaagaatggaagagatttttaggagaatacaaaaata

SEQ ID NO: 25 - DNA, 235 bp, Canis familiaris
ccaaataaatgcagcctggaatctgcatgcagcctccgtttctgccgtagcaatttaaaggagaatcttgctgctcaggcagaagaaattcaatgacctggtggagataatgaaagagctttatacttttaatcagatattgactgattactgtaatactctcctgtagcaatagctgtaggcatgagaaatagggaaaaaaatggaagagattttcaggaggatatgaagaaaa

SEQ ID NO: 26 - DNA, 241 bp, Mus sp.
caccatgaaaactcagtccagacctcacacaaaaatgatagtcactgctgcaacaatttaaaagaaaaacccatgctgctcagcagggtacttcaatcacctcctggtgatatagagcctcagactgctaatctgatattgattgtttgctgtaatactctcctatagcagtagctgcaagcataagaagcagaggtagaagatccgggaaggttagaaataaacacgtgggtgagactta

SEQ ID NO: 27 - DNA, 233 bp, Rattus sp.
acaccatgaaaactcagtccagacctcacacaaaatgatagtcgctgcagcagcaatttaaaagaaaaatgtgcttctcagcagggtactttaatcacctcctggagttacagggcttcagaatgctaatctgatatcgatggtttgccataatactttccggcagcagtagctgcaggcatagaactagaggaagagcaggggagggtatgaaataaatgcatgtttgagac

SEQ ID NO: 28 - DNA, 248 bp, Gallus gallus
cacaataagtatcagagcttgggagcaaggtgggggtttccccacggcaaggtaaaatattgtcttcccgttatggctgcagcaattcaatctgggcttgaaggtaatgaaagagcttcattcatttaatcagatattgattgattgctataatatactatgtcaacaattgctgcaagggaacaagggctggctgaaataaatcagattccagctggctctgttagaagaagtaaaaattatgccaa

SEQ ID NO: 29 - DNA, 216 bp, Homo sapiens
ctgctgcagcaattcaaaggagaatcttgctgttcgggcagaagaaattcaatcaccttgtggagataatgaaaaagcttcatacttttaatcagatattgatcgattaccataatattctcccatagcaatagctgcaggcataagaaacggaaagaatggaagagatttttaggagaatacaaaaataaataagtatttgagacttagatactg

SEQ ID NO: 30 - DNA, 218 bp, Canis familiaris
ctgccgtagcaatttaaaggagaatcttgctgctcaggcagaagaaattcaatgacctggtggagataatgaaagagctttatacttttaatcagatattgactgattactgtaatactctcctgtagcaatagctgtaggcatgagaaatagggaaaaaaatggaagagattttcaggaggatatgaagaaaataagtgtttgagacttggaaactg

SEQ ID NO: 31 - DNA, 208 bp, Mus sp.
gcaattaccttaaaagctttgatgtatatttaattgattatcattcaaaggaaattatttctgtattggagagattatctgatattcatagccatttccactgactgattgcaaacttgtctacccaacggtaaacagaaagccaatgaggtgaatttattacttatgaattatgctgaaaagataggtgaagaaggaatataataat

SEQ ID NO: 32 - DNA, 209 bp, Rattus sp.
gcttccatgcaattaccttaaaagctttgatgtatatttaattgattatcattcaatggaaattattcctgtacttgagagattatgtgatatttctggccacttctactgactgattgcaaacttgtctgccagagggtatacagaaagccaatgaggtgaatttattacttatgaattatgctgaaaagataggtgaaaaggtatat

SEQ ID NO: 33 - DNA, 201 bp, Gallus gallus
ccacggcaaggtaaaatattgtcttcccgttatggctgcagcaattcaatctgggcttgaaggtaatgaaagagcttcattcatttaatcagatattgattgattgctataatatactatgtcaacaattgctgcaagggaacaagggctggctgaaataaatcagattccagctggctctgttagaagaagtaaaaatta

SEQ ID NO: 34 - DNA, 20 bp, Artificial sequence
  Synthetic sequence Oligo MHR1_FOR
cagaaaactagtcaggtgtg

SEQ ID NO: 35 - DNA, 23 bp, Artificial sequence
  Synthetic sequence Oligo MHR1_REV
cagaaccggttaaagttattagt

SEQ ID NO: 36 - DNA, 27 bp, Artificial sequence
  Synthetic sequence Oligo MHR2_FOR
ggttgggccctttaaagaacattctaa

SEQ ID NO: 37 - DNA, 27 bp, Artificial sequence
  Synthetic sequence Oligo MHR2_REV
aagactaagaattcaaactcatgttaa

SEQ ID NO: 38 - DNA, 22 bp, Artificial sequence
  Synthetic sequence Oligo ECR1_FOR
tcaagataattttgccatgctg

SEQ ID NO: 39 - DNA, 21 bp, Artificial sequence
  Synthetic sequence Oligo ECR1_REV
gatcggtatagcaaaccacca

SEQ ID NO: 40 - DNA, 20 bp, Artificial sequence
  Synthetic sequence Oligo ECR2_FOR
ttttgggagaatggaagtgg

SEQ ID NO: 41 - DNA, 20 bp, Artificial sequence
  Synthetic sequence Oligo ECR2_REV
tggcttggggtaatcttttt